(19) United States
(12) Patent Application Publication
(10) Pub. No.: US 2011/0112020 A1
(43) Pub. Date: May 12, 2011

(54) MEASUREMENT OF G-PROTEIN mRNA IN THE DIAGNOSIS OF GROWTH HORMONE INSUFFICIENCY

(75) Inventors: Jill D. Jacobson, Kansas City, MO (US); Mark Clements, Kansas City, MO (US)

(73) Assignee: THE CHILDREN'S MERCY HOSPITAL, Kansas City, MO (US)

(21) Appl. No.: 12/771,377

(22) Filed: Apr. 30, 2010

Related U.S. Application Data
(60) Provisional application No. 61/174,386, filed on Apr. 30, 2009.

(51) Int. Cl. A61K 38/25; C12Q 1/68; A61P 5/06

(52) U.S. Cl. 514/11.2; 435/6

(57) ABSTRACT

The present invention is directed towards a diagnostic test, treatment, and monitoring of treatment for growth hormone abnormalities including Growth Hormone Deficiency (“GHD”) as well as a kit comprising the necessary components of the present invention. G-protein expression and levels of mRNA are evaluated to determine if a patient has GHD or if treatment for GHD is effective. A G-protein agonist or antagonist is used to treat GHD by bringing G-protein expression and levels of mRNA into the normal range.

### RELATED APPLICATIONS

This application claims the benefit of provisional Application No. 61/174,386, filed Apr. 30, 2009, the teaching and contents of which are hereby incorporated by reference.

### SEQUENCE LISTING

This application contains a sequence listing in paper format and in computer readable format, the teachings and content of which are hereby incorporated by reference.

### BACKGROUND

Growth hormone deficiency (GHD) in childhood has major clinical consequences. GHD is often associated with other pituitary hormone deficiencies. GHD children may require multiple hormonal replacement treatments and close clinical follow-up to optimize their outcome. Growth hormone (GH) stimulation testing, as currently conducted, is not a reliable diagnostic tool. A substantial proportion of normal children are incorrectly diagnosed as having idiopathic GHD. Conversely, significant numbers of patients exhibit “normal” responses to provocative testing, but continue to grow poorly. Currently, GHD is diagnosed using 2 separate provocative stimulation tests. This testing costs in the neighborhood of $4,000.00, necessitates i.v. placement, and takes 4 hours to perform. Currently, most endocrinologists use a cut-off of 10 ng/ml to diagnose GHD. A recent study demonstrated very little correlation between 2 sets of growth hormone testing results when the test was repeated 2 weeks apart. Insurance companies will use a value of ≧10 ng/ml to deny GH treatment. In the past, adult GHD was diagnosed using a commercially available preparation, growth hormone releasing hormone (GHRH) Geref®(Serono Laboratories, Rockland, Mass.). Geref® has also been used to stimulate the pituitary gland to release growth hormone. The company stopped manufacturing this in October of 2008. No product is available to take its place.

What is needed in the art is a more accurate and less expensive diagnostic test for GHD. A new test that would correctly diagnose GH insufficiency and obviate the need for cumbersome provocative testing would be welcome to endocrinologists. What is further needed are methods and compositions for modifying GH levels. What is still further needed are methods, compositions, and treatments for GH insufficiency.

### SUMMARY OF THE INVENTION

The present invention overcomes the problems of the prior art and provides a distinct advantage and improvement in the state of the art. The disclosure of the present invention provides a diagnostic test for GH abnormalities, including GHD, utilizing analysis of G-protein expression levels. Additionally, the present invention provides methods for modifying GH expression and, in preferred forms, provides a treatment for GH abnormalities, including GHD. Further, the present invention provides a method of regulating and monitoring efficacy of treatment for GH abnormalities, including GHD. The embodiments of the present invention are based on the surprising finding that patients with GHD have abnormally low levels of mRNA for a stimulatory G protein, preferably Gαq. This is the first disclosure linking G-protein expression with GHD. Using levels of G-protein expression, both childhood and adult GHD can be diagnosed.

The diagnostic test of the present invention can be used to determine if a patient has a GH abnormality, such as GHD, by completing the steps of analyzing G-protein expression levels and comparing those levels to patients who do not have GHD or by comparing the G-protein levels to those normally found in a patient who does not have GHD. If low levels (relative to normal or standard levels in individuals that do not have GHD) of G-protein expression are found, this would indicate that a patient has GHD. Preferably, stimulatory G-protein expression levels are measured for diagnosing GHD. Preferably, the stimulatory G-protein is a stimulatory G-protein alpha subunit. Stimulatory G-protein alpha subunits that are preferably used include, but are not limited to, Gα11, Gαq, Gαs, Gα13, Gα14, Gα15, and combinations thereof. In a most preferred embodiment, the G-protein is Gαq or Gαs.

For purposes of the present invention, “lower G-protein expression levels and levels of mRNA in patients with GHD” refers to G-protein expression levels and levels of mRNA in patients with GHD that are at least 10% lower, more preferably, at least 20% lower, still more preferably, at least 30% lower more preferably, at least 40% lower, even more preferably, at least 50% lower, more preferably, at least 60% lower, still more preferably, at least 70% lower, and most preferably, at least 80% lower, than the G-protein expression levels and levels of mRNA in patients without GHD or normal controls.

The diagnostic test for purposes of the present invention utilizes an assay for determining the level of G-protein expression in a patient. Any assay that quantifies G-proteins will work for purposes of the present invention. In a preferred embodiment, i-cycler PCR normalized to a housekeeping gene is used to quantitate G-protein expression. Some examples of labs which perform assays quantifying G-proteins are BioVision, Inc. (Mountain View, Calif.) and EMD Biosciences, Inc. (San Diego, Calif.).

In a preferred embodiment, Gαq levels are analyzed for purposes of the diagnostic test. The diagnostic test can also comprise a kit of parts. The kit preferably includes a receptacle for collecting a sample from a patient, components used to complete an assay to quantitate G-protein expression, a set of values from normal controls for comparison, and an instruction manual.

A method of treatment for modulating GH abnormalities, including GHD, is also disclosed in the present invention. The method of treatment generally comprises the administration of a G-protein agonist or antagonist to a patient in need thereof such that the patient's G-protein levels are restored to a normal range. In cases where G-protein levels are low, as set forth above, a G-protein agonist is preferably administered. Conversely, in cases where G-protein levels are high in comparison to normal levels, a G-protein antagonist is preferably administered. Preferably, the agonist or antagonist is directed towards expression of Gαq. G-protein agonists and antagonists are well known in the art and those of skill in the art will be able to determine and select appropriate agonists or antagonists. For example, G-protein agonists include, but are not limited to, Growth hormone releasing hormone (GHRH) (e.g., brand name Geref® (Serono Laboratories, Rockland, Mass.); Gonadotropin-releasing (GnRH) (e.g., brand name Lupron® (Abbott Laboratories, Abbott Park, Ill.)); Corticotropin-releasing hormone (CRH); Adrenocorticotropic hormone (ACTH) (e.g., brand name Cortrosyn® (Amphastar Pharmaceuticals, Inc., Rancho Cucamonga, Calif.); Follicle stimulating hormone (FSH) (e.g., brand name Follistim® (Schering-Plough, Kenilworth, N.J.); Luteinizing hormone (LH); and human chorionic gonadotropin (hCG). Preferably, G-protein antagonists are G-protein antagonist peptides. Examples of G-protein antagonist peptides include, but are not limited to, GPAnt-2(SEQ ID NO: 1: pGlu-Gln-D-Trp-Phe-D-Trp-D-Trp-Met-NH₂) (e.g. Sigma-Aldrich product # G9541-1M (Sigma-Aldrich, St. Louis, Mo.); and 2′3′-dialdehyde analogue (oGTP) (Nanoff, Christian et. al. “2′,3′-Dialdehyde GTP as an Irreversible G Protein Antagonist” The Journal of Biological Chemistry, Vol. 269, No. 50, December 16, pp. 31999-32007, 1994, the contents and teachings of which are incorporated herein by reference).

In another embodiment of the present invention, a method of regulating treatment of GH abnormalities, including GHD, is disclosed. The method comprises monitoring G-protein expression in patients to determine if treatment is working effectively. If G-protein expression levels are high, then an amount or an increased amount of G-protein antagonist may be administered. If G-protein expression levels are low, then an amount or increased amount of G-protein agonist may be administered. Preferably, high or low G-protein expression levels are in comparison to the G-protein expression levels in a patient that does not have a GH abnormality, including GHD. Preferably, levels of G-protein expression in normal controls would be assayed for purposes of comparing those levels to the G-protein expression levels of patients suspected of having GHD. This method also can help physicians determine if treatment for a GH abnormality, including GHD, is effective.

In an embodiment where the effectiveness of treatment for a GH abnormality, including GHD, is being analyzed, the method for monitoring or evaluating effectiveness of treatment preferably includes the steps of determining the G-protein expression levels or levels of mRNA in a patient prior to the administration of a G-protein agonist or antagonist; administering to the patient a G-protein agonist or antagonist; analyzing the amount of G-protein expression or mRNA levels in the patient; and comparing the levels to the controls or a standard level of G-protein expression. If the G-protein expression or mRNA levels are higher than they were prior to administration of the G-protein agonist, then it can be determined that the treatment is working. If the G-protein expression or mRNA levels are lower than they were prior to administration of the G-protein antagonist, then it can be determined that the treatment is working. This method can also be used to determine appropriate dosing regiments for patients with GHD. In such cases, the method generally comprises the steps of determining the G-protein expression levels or levels of mRNA in a patient prior to the administration of a G-protein agonist; administering to the patient a G-protein agonist; analyzing the amount of G-protein expression or mRNA levels in the patient; and comparing the levels to the controls or a standard level of G-protein expression.

GH stimulation tests were performed on healthy control subjects and subjects previously diagnosed with childhood GH deficiency. Growth hormone releasing hormone (GHRH), Sermorelin Acetate, sold commercially as Geref® (Serono Laboratories, Rockland, Mass.—no longer produced) was used as a provocative agent. Results showed that patients with childhood growth hormone deficiency displayed abnormally low levels of mRNA for a stimulatory G protein, Gαq. No overlap exists in mRNA levels between healthy control subjects and patients with GHD.

G proteins have not been a major focus of study in the field of growth. Much growth research focuses on the transport of GH and IGF-1, their receptors, and their post-receptor pathways. However, G proteins may play an important role in growth at two major levels: 1) Secretion of growth hormone by somatotropes is stimulated by growth hormone releasing hormone (GHRH) via its G protein-coupled receptor. 2) Numerous growth factors active at the growth plate are G protein-coupled hormones. Such G protein-coupled hormones include PTH, PTHrP, transforming growth factor-β (TGF-β), fibroblast growth factors (FGFs), and vascular endothelial growth factor (VEGF). Patients with heterozygous loss-of-function mutations of the GNAS1 gene, encoding the major stimulatory G protein, Gαs, display a 70% incidence of growth hormone deficiency (and an even higher incidence of short stature). Somatic gain-of-function mutations in the GNAS1 gene are associated with acromegally.

It was a finding of the present invention that GH responses to GHRH in humans correlate with transcriptional regulation of these G proteins in peripheral blood mononuclear cells (PBMCs). An investigation was performed to determine whether growth hormone deficient participants displayed abnormalities in expression of stimulatory G protein mRNA in peripheral blood cells after GHRH administration. Growth hormone testing was performed using GHRH and arginine in 6 young adults with childhood growth hormone deficiency and in healthy male and female control participants (n=20). Serial GH levels were measured after administration of standard doses of GHRH. Gαq and Gαs mRNA content in peripheral blood were quantitated by i-cycler PCR and normalized to a housekeeping gene. All data were expressed as a percentage of control mRNA consisting of pooled mRNA from healthy adult male and female participants. It was surprisingly found that participants with childhood growth hormone deficiency exhibited significantly lower Gαq mRNA expression compared to healthy control participants at all time points tested. Thus, the present invention is particularly suited for treating patients with GHD that exhibit at least 10% lower Gαq mRNA expression, more preferably, at least 20% lower Gαq mRNA expression, still more preferably, at least 30% lower Gαq mRNA expression, more preferably, at least 40% lower Gαq mRNA expression, even more preferably, at least 50% lower Gαq mRNA expression, more preferably, at least 60% lower Gαq mRNA expression, still more preferably, at least 70% lower Gαq mRNA expression, and most preferably, at least 80% lower Gαq mRNA expression. Importantly, even at baseline, GH deficient participants expressed significantly lower Gαq mRNA levels compared to healthy participants (p=0.02; FIG. 1). Similarly, the patients with GHD that exhibit at least 10% lower Gαq mRNA levels, more preferably, at least 20% lower Gαq mRNA levels, still more preferably, at least 30% lower Gαq mRNA levels, more preferably, at least 40% lower Gαq mRNA levels, even more preferably, at least 50% lower Gαq mRNA levels, more preferably, at least 60% lower Gαq mRNA levels, still more preferably, at least 70% lower Gαq mRNA levels, and most preferably, at least 80% lower Gαq mRNA levels are particularly suited for treatment using the methods of the present invention. In another surprising aspect, fifteen minutes after injection of GHRH, GH deficient participants expressed significantly lower Gαq mRNA levels compared to control participants (p=0.008). Similar, though much less pronounced, differences were seen in Gαs mRNA expression (FIG. 3). A correlation was seen between peak Gαq mRNA levels and peak GH responses to GHRH (data not shown; r²=0.33).

It was found that peripheral blood immune cells express the GHRH receptor. There are multiple reports in the literature indicating that there are not only GHRH receptors on blood mononuclear cells, but that these receptors are functional. GHRH and GHRH antagonist (MZ-4-71) exert opposite effects on interferon-gamma secretion from human peripheral blood mononuclear cells in vitro. There is evidence to suggest that GHRH plays a role in the immune activation of experimental autoimmune disease.

For purposes of the present invention, a “G-protein abnormality” refers to an increased or decreased level of G-protein expression in an individual in comparison to an individual that has a normal level of G-protein expression. GHD is an example of a G-protein abnormality.

The present invention provides a method of diagnosing growth hormone abnormalities comprising the steps of: analyzing the G-protein or mRNA levels in an individual; and diagnosing growth hormone abnormalities based on said analyzed G-protein or mRNA levels. The method preferably includes analysis of the G-protein or mRNA levels by obtaining a fluid sample from an individual and using the fluid sample for to analyze the G-protein or mRNA levels. In another embodiment, the method further comprises the step of comparing the analyzed G-protein or mRNA levels with G-protein or mRNA levels of individuals known to not have any growth hormone abnormality. Preferably, the G-protein levels are determined from the expression of mRNA for a stimulatory G-protein. Preferably the G-protein is Gαq, Gαs, and combinations thereof. In a further embodiment, the growth hormone abnormality is selected from the group consisting of Growth Hormone Deficiency or Growth Hormone Excess. The method preferably includes the further step of diagnosing an individual with Growth Hormone Deficiency when the analyzed G-protein or mRNA levels are lower in the individual when compared to G-protein or mRNA levels in an individual or group of individuals that do not have a growth hormone abnormality. Preferably, the method includes the step of administering a G-protein agonist to an individual diagnosed with Growth Hormone Deficiency. The G-protein agonist is preferably selected from the group consisting of Gαq agonists, Gαs agonists, and combinations thereof.

The present invention also provides a method of treating Growth Hormone Deficiency, comprising the step of administering a G-protein agonist to an individual diagnosed with or suspected or having Growth Hormone Deficiency. Preferably, the G-protein agonist is selected from the group consisting of Gαq agonists, Gαs agonists, and combinations thereof. The method preferably includes that step of measuring the level of G-protein in the patient after the step of administering a G-protein agonist. Additionally, the method preferably includes the step of comparing the G-protein levels with G-protein levels of an individual that does not have a growth hormone abnormality. Further, the method optionally comprises a further step of modifying the amount of the G-protein agonist administered to the individual when the G-protein level of the individual diagnosed with or suspected of having Growth Hormone Deficiency is low in comparison to the G-protein level of an individual that does not have a growth hormone abnormality. Preferably, the amount G-protein agonist administered to the individual is increased. It is preferable that the G-protein levels are measured by the expression of mRNA in a stimulatory G-protein. Preferably, the stimulatory G-protein is selected from the group consisting of Gαq, Gαs, and combinations thereof. In a preferred embodiment, the G-protein level is determined by analysis of G-protein expression levels or levels of mRNA.

The present invention further provides for a kit comprising an assay for G-protein levels, instructions for comparing and levels to individuals that are known not to have a growth hormone abnormality, and a container to hold the assay and the instructions. The assay preferably includes a fluid sample container and components designed to measure a level selected from the group consisting of G-protein expression levels, G-protein mRNA levels, and combinations thereof.

### BRIEF DESCRIPTION OF FIGURES

FIG. 1 is a graph illustrating a comparison of Gαq mRNA expression in control subjects versus GH deficient subjects;

FIG. 2 is a graph illustrating a comparison of Gαs mRNA expression in control subjects versus GH deficient subjects;

FIG. 3 is a graph illustrating a comparison of Gαq mRNA expression in control subjects versus GH deficient subjects, where some subjects were undergoing GHRH stimulation and some were not; and

FIG. 4 is a graph illustrating a comparison of Gαq mRNA expression in control subjects versus GH deficient subjects undergoing GHRH stimulation testing.

### DETAILED DESCRIPTION

The following examples describe representative embodiments of the present invention. It is understood that these examples are provided for representative purposes only and nothing herein shall be deemed a limitation on the overall scope of the invention.

### Example 1

### Materials and Methods

Subjects. Subjects consisted of 6 male patients previously diagnosed with childhood GH deficiency who were undergoing GHRH stimulation testing for adult GH deficiency. No patient had any additional pituitary abnormalities. Controls consisted of age-matched, sex-matched healthy controls with no endocrine abnormalities. After IRB approval and screening for inclusion/exclusion criteria, informed consent was obtained from each participant. Participants fasted overnight, and a peripheral IV was placed the next morning. The test was initiated between 8 am and 10 am in all cases. Normal saline was infused IV at TKO for the duration of the test. Baseline blood for RNA isolation from peripheral blood mononuclear cells (PBMC) was drawn at t=0 minutes. Geref was immediately given IV at a dose of 1 microgram/kg via IV push over 2 minutes, followed by arginine infusion over 30 minutes (0.5 gm/kg dose of 10%).

PBMC Collection and RNA Isolation. Blood was drawn after obtaining informed consent. 30 mL of whole blood was collected directly into Tempus tubes (ABI, Foster City, Calif.) in order to prevent RNA degradation, as well as alterations in gene expression pattern between blood collection and blood processing. PBMC RNA was isolated using 5-Prime human blood extraction kit (Fisher Scientific, Pittsburgh, Pa.) as directed.

RNA. RNA was quantitated on an F1×800 fluorescence microplate reader using the Ribogreen assay (Invitrogen, Carlsbad, Calif.). 1 μg RNA was used for cDNA preparation. Residual DNA was digested with DNAase I (Invitrogen, Carlsbad, Calif.) for 15 min at 65° C., then quenched with 2.5 mM EDTA. 10 mM dNTP and 0.5 ug/ml oligo (dT) were added to ice-cooled DNase-treated RNA samples (1 μg), with subsequent incubation at 65° C. for 5 min. Samples were again cooled on ice, and reverse transcription was performed using the SuperScript II Reverse Transcription Kit (Invitrogen, Carlsbad, Calif.) for 50 min at 42° C., according to manufacturer instructions. RNaseOUT recombinant RNase inhibitor was added to all reactions. Samples were subsequently treated with RNase H, and DEPC-treated water was added to make a final volume of 50 μl cDNA.

RT-PCR. Real-time RT-PCR was performed using the SYBR Green PCR kit (Bio-Rad, Hercules, Calif.). Human Gαs, Gαq, and GAPDH mRNA sequences were obtained from the Gene Bank database and primers were designed as follows: Gαq sense: 5′-GAT GTT CGT GGA CCT GAA CC-3′ (SEQ ID NO. 2); Gαq antisense: 5′-CAA CTG GAC GAT GGT GTC CT-3′ (SEQ ID NO. 3); Gαs sense: 5′-TCT ACC GGG CCA CGC ACC GC-3′ (SEQ ID NO. 4); Gαs antisense: 5′-GCA GGA TCC TCA TCT GCT TC-3′ (SEQ ID NO. 5); GAPDH sense: 5′-TGA CAA CTT TGG TAT CGT GGA AGG-3′ (SEQ ID NO. 6); GAPDH antisense: 5′-AGG GAT GAT GTT CTG GAG AGC C-3′ (SEQ ID NO. 7). Real time quantitative PCR was performed on the iCycler (Bio-Rad, Hercules, Calif.). The following parameters were used for the RT-PCR program: 95° C. at 3 min; 35 cycles of 95° C. at 20 sec, 56° C. at 20 sec, 72° C. for 20 sec; 95° C. for 1 min; and 55° C. at 1 min. The AA-CT method of relative quantification was used. Data are expressed as a percent of control, which was from PMBC RNA from a pool of normal adult human blood.

GH measurement. GH was measured by chemiluminescence immunoassay (DPC, Los Angeles, Calif.)

Statistics. Data from patients and controls were compared by a Student's unpaired t test.

### Results and Conclusions

The G protein levels were found to be lower in patients with GHD than those patients who did not have GHD

### Example 2

The objective of this study was to determine whether growth hormone deficient subjects displayed abnormalities in expression of stimulatory G protein mRNA in PBMCs after GHRH administration. Sex differences were also sought.

### Materials and Methods

After obtaining informed consent, growth hormone testing was performed using GHRH and arginine in 6 young adults with childhood growth hormone deficiency and in healthy male and female control participants (n=20). GH deficient participants received no growth hormone for at least two months prior to testing. GH deficient subjects ranged in age from 15 yrs 2 months to 17 years 10 months. Control subjects ranged in age from 15 years 11 months to 22 years 2 months. Serial GH levels were measured after administration of standard doses of GHRH. Gαq and Gαs mRNA content in PBMC's were quantitated by i-cycler PCR and normalized to a housekeeping gene. All data are expressed as a percentage of control mRNA consisting of pooled mRNA from healthy adult male and female subjects.

### Results and Conclusions

Participants with childhood growth hormone deficiency exhibited significantly lower Gαq mRNA expression compared to healthy control participants at all time points tested. At baseline, GH deficient subjects expressed Gαq mRNA levels that were 25.3±5.2% of control compared to healthy participants whose Gαq mRNA levels were 303.8±60.2% of control (p=0.02). Fifteen minutes after injection of GHRH, GH deficient participants expressed Gαq mRNA levels of 89.8±35.0% of control compared to 665.3±108.1% in control subjects (p=0.008). Similar, though less pronounced, differences were seen in Gαs mRNA expression. Peak Gαq mRNA levels correlated positively with peak GH responses to GHRH in males (r=0.58). No major sex differences in G protein mRNA responses were identified.

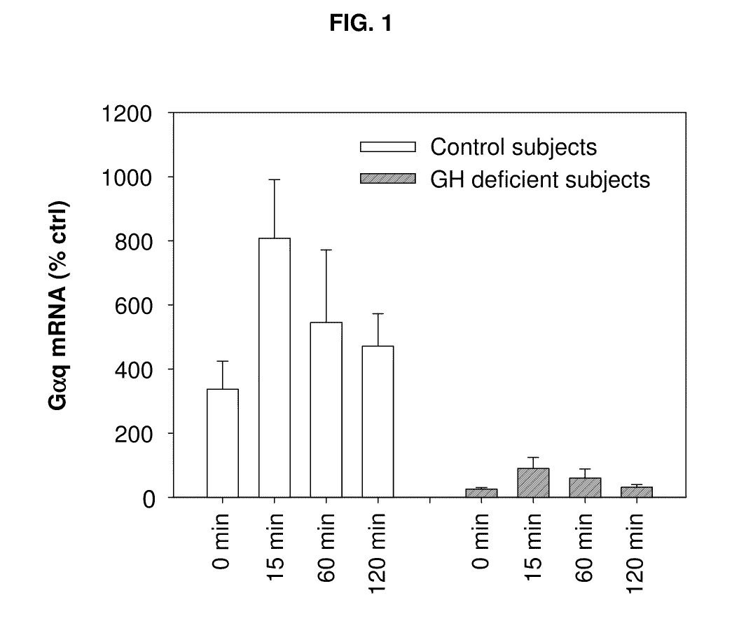

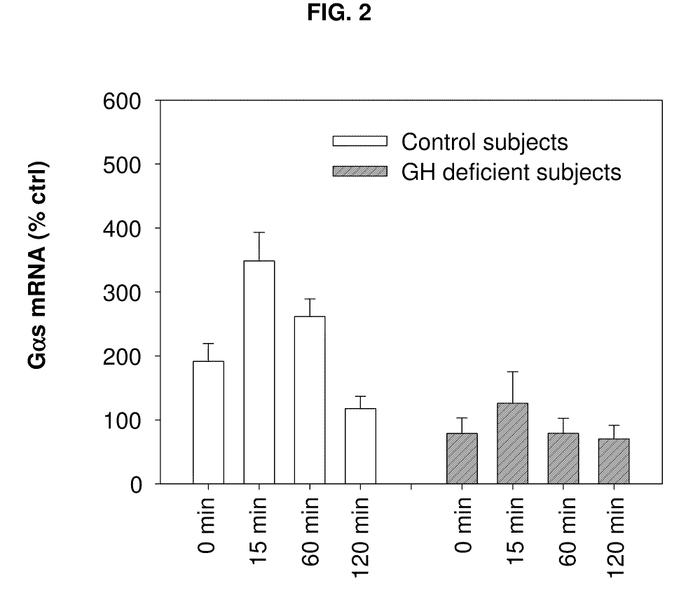

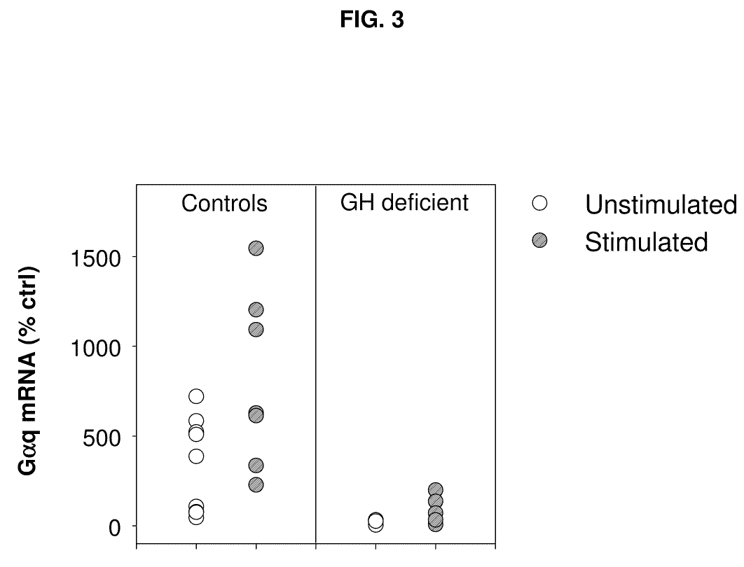

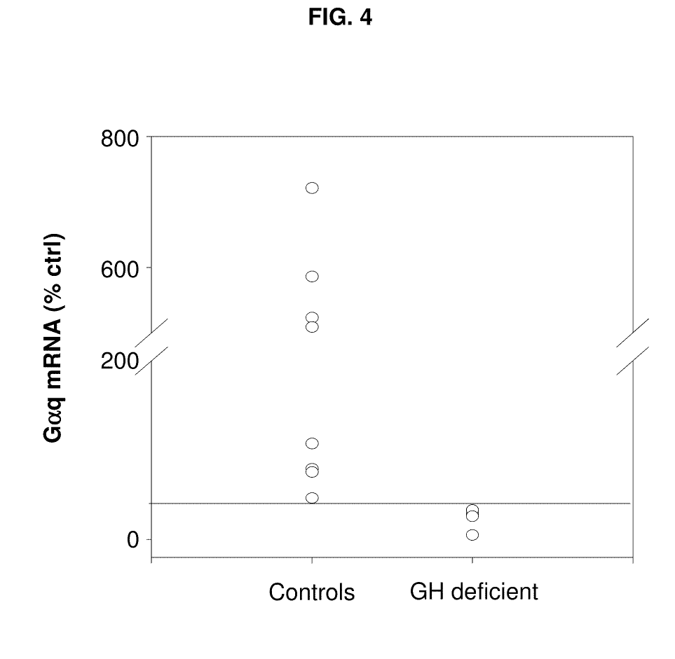

FIG. 1 shows Gαq mRNA expression in healthy young male adult control subjects and in young adults preciously diagnosed with childhood GH deficiency undergoing GHRH stimulation testing. Differences exist even at baseline (p−0.02). FIG. 2 illustrates Gαs mRNA expression in healthy young male adult control subjects and in young adults preciously diagnosed with childhood GH deficiency undergoing GHRH stimulation testing. FIG. 3 illustrates Gαq mRNA expression in healthy young male adult control subjects and in young adults preciously diagnosed with childhood GH deficiency undergoing GHRH stimulation testing. FIG. 4 shows Gαq mRNA expression in healthy young male adult control subjects and in young adults preciously diagnosed with childhood GH deficiency undergoing GHRH stimulation testing. No overlap exists at the 0 minute time point.

### CONCLUSIONS

Stimulatory G protein pathways deserve greater attention in investigation of the etiology of short stature. G-protein expression levels were found to be lower in those patients with GHD.

### REFERENCES

The teaching and content of the following references are incorporated by reference herein.
1. Wilson, D. M. and J. Frane, A brief review of the use and utility of growth hormone stimulation testing in the NCGS: do we need to do provocative GH testing? Growth Horm IGF Res, 2005. 15 Suppl A: p. S21-5.
2. Capdevila, J. and J. C. Izpisua Belmonte, Patterning mechanisms controlling vertebrate limb development. Annu Rev Cell Dev Biol, 2001. 17: p. 87-132.
3. Germain-Lee, E. L., Short stature, obesity, and growth hormone deficiency in pseudohypoparathyroidism type 1a. Pediatr Endocrinol Rev, 2006. 3 Suppl 2: p. 318-27.
4. Germain-Lee, E. L., et al., Growth hormone deficiency in pseudohypoparathyroidism type 1a: another manifestation of multihormone resistance. J Clin Endocrinol Metab, 2003. 88(9): p. 4059-69.
5. Mantovani, G., et al., Parental origin of Gsalpha mutations in the McCune-Albright syndrome and in isolated endocrine tumors. J Clin Endocrinol Metab, 2004. 89(6): p. 3007-9.
6. Hayward, B. E., et al., Imprinting of the G(s)alpha gene GNAS1 in the pathogenesis of acromegaly. J Clin Invest, 2001. 107(6): p. R31-6.
7. Siejka, A., et al., Effect of growth hormone-releasing hormone (GHRH) and GHRH antagonist (MZ-4-71) on interferon-gamma secretion from human peripheral blood mononuclear cells in vitro. Neuropeptides, 2004. 38(1): p. 35-9.
8. Ikushima, H., M. Kanaoka, and S. Kojima, Cutting edge: Requirement for growth hormone-releasing hormone in the development of experimental autoimmune encephalomyelitis. J Immunol, 2003. 171(6): p. 2769-72.

1. A method of diagnosing growth hormone abnormalities comprising the steps of:
analyzing the G-protein or mRNA levels in an individual; and
diagnosing growth hormone abnormalities based on said analyzed G-protein or mRNA levels.

2. The method of claim 1, wherein said analyzing step includes the step of obtaining a fluid sample from said individual and using said fluid sample for said analyzing step.

3. The method of claim 1, further comprising the step of comparing said analyzed G-protein or mRNA levels with G-protein or mRNA levels of individuals known to not have any growth hormone abnormality.

4. The method of claim 1, wherein said G-protein levels are determined from the expression of mRNA for a stimulatory G-protein.

5. The method of claim 1, wherein said stimulatory G-protein is selected from the group consisting of Gαq, Gαs, and combinations thereof.

6. The method of claim 1, said growth hormone abnormality being selected from the group consisting of Growth Hormone Deficiency and Growth Hormone Excess.

7. The method of claim 6, further comprising the step of diagnosing an individual with Growth Hormone Deficiency when said analyzed G-protein or mRNA levels are lower in said individual when compared to G-protein or mRNA levels in an individual or group of individuals that do not have a growth hormone abnormality.

8. The method of claim 7, further comprising the step of administering a G-protein agonist to an individual diagnosed with Growth Hormone Deficiency.

9. The method of claim 8, said G-protein agonist being selected from the group consisting of Gαq agonists, Gαs agonists, and combinations thereof.

10. A method of treating Growth Hormone Deficiency, comprising the step of administering a G-protein agonist to an individual diagnosed with or suspected of having Growth Hormone Deficiency.

11. The method of claim 10, wherein said G-protein agonist is selected from the group consisting of Gαq agonists, Gαs agonists, and combinations thereof.

12. The method of claim 10, further comprising the step of measuring the level of G-protein in the patient after said step of administering a G-protein agonist.

13. The method of claim 12, further comprising the step of comparing said G-protein level with the G-protein levels of an individual that does not have a growth hormone abnormality.

14. The method of claim 13, further comprising the step of modifying the amount of said G-protein agonist administered to said individual when the G-protein level of the individual diagnosed with or suspected of having Growth Hormone Deficiency is low in comparison to the G-protein level of an individual that does not have a growth hormone abnormality.

15. The method of claim 14, wherein said amount of G-protein agonist administered to the individual is increased.

16. The method of claim 12, wherein said G-protein levels are measured by the expression of mRNA of a stimulatory G-protein.

17. The method of claim 16, wherein said stimulatory G-protein is selected from the group consisting of Gαq, Gαs, and combinations thereof.

18. The method of claim 12, wherein said G-protein level is determined by analysis of G-protein expression levels or levels of mRNA.

19. A kit comprising an assay for G-protein levels, instructions for comparing said levels to individuals that are known to not have a growth hormone abnormality, and a container to hold said assay and said instructions.

20. The method of claim 19, wherein said assay includes a fluid sample container and components designed to measure a level selected from the group consisting of G-protein expression levels, G-protein mRNA levels, and combinations thereof.


SEQUENCE LISTING (7 sequences)

SEQ ID NO: 1 - protein, 7 aa, Homo sapiens
  MISC_FEATURE (3)..(6): All Trp′s are in the D configuration
EQWFWWM

SEQ ID NO: 2 - DNA, 20 bp, Homo sapiens
gatgttcgtggacctgaacc

SEQ ID NO: 3 - DNA, 20 bp, Homo sapiens
caactggacgatggtgtcct

SEQ ID NO: 4 - DNA, 20 bp, Homo sapiens
tctaccgggccacgcaccgc

SEQ ID NO: 5 - DNA, 20 bp, Homo sapiens
gcaggatcctcatctgcttc

SEQ ID NO: 6 - DNA, 24 bp, Homo sapiens
tgacaactttggtatcgtggaagg

SEQ ID NO: 7 - DNA, 22 bp, Homo sapiens
agggatgatgttctggagagcc